(19) United States
(12) Patent Application Publication
(10) Pub. No.: US 2026/0096982 A1
(43) Pub. Date: Apr. 9, 2026

(54) PLANT-BASED ANTI-WRINKLE COMPOSITION AND METHOD FOR PREPARING SAME

(71) Applicant: Inor Bio Co., Ltd., Hwaseong-si (KR)

(72) Inventors: Jong Soo CHO, Pyeongtaek-si (KR); Mi Jin KANG, Pyeongtaek-si (KR); Eun JO, Hwaseong-si (KR); Pu Reum CHO, Hwaseong-si (KR)

(21) Appl. No.: 19/008,756

(22) Filed: Jan. 3, 2025

(30) Foreign Application Priority Data
Oct. 8, 2024 (KR) 10-2024-0136546

(51) Int. Cl. A61K 8/9789; A01G 31/00; A61K 8/44; A61K 8/46; A61K 8/49; A61K 36/48; A61Q 19/08

(52) U.S. Cl. CPC A61K 8/9789; A01G 31/00; A61K 8/44; A61K 8/46; A61K 8/4913; A61K 8/4946; A61K 36/48; A61Q 19/08; A61K 2236/37; A61K 2236/39; A61K 2236/53; A61K 2800/81

(57) ABSTRACT

The present disclosure provides a plant-based anti-wrinkle composition comprising plant-based amino acids extracted from sprouted plant sprouts, wherein the sprouted plant sprout is hydroponically sprouted by irradiating red light and blue light in the presence of zinc-sulfur solution manufactured from sphalerite. The present disclosure has the advantage of highly efficiently extracting only high-quality plant-based amino acids from sprouted sprouts to have excellent moisturizing and anti-wrinkle effects and to form skin with elasticity, vitality, and volume.

### CROSS-REFERENCE TO RELATED APPLICATION(S)

This application claims priority to Korean Patent Application No. 10-2024-0136546 filed in the Korean Intellectual Property Office on Oct. 8, 2024, the disclosure of which is incorporated by reference herein in its entirety.

### TECHNICAL FIELD

The present disclosure relates to a plant-based wrinkle-improving composition and a method for producing the same, and more specifically, to a plant-based wrinkle-improving composition and a method for producing the same, which reduces wrinkles, which are damaged skin tissues, by germinating plant seeds and extracting plant-based amino acids, thereby activating collagen proliferation and regeneration when applied to the skin.

### DISCUSSION OF RELATED ART

The skin is the body's most basic defense against irritation from contact with the external environment, and it is an important organ for expressing external beauty.

In the modern world, people are increasingly taking care of their skin because it is not only a standard of judgment that symbolizes youth and health, but also a competitive advantage that expresses confidence and affects work performance.

However, as people age, the secretion of various hormones that regulate metabolism decreases, the function of immune cells and the activity of cells decreases, and the biosynthesis of immune proteins and biocomponent proteins necessary for life decreases, and externally, physical and chemical stimuli and stress caused by the increase of ultraviolet rays, free radicals, and active oxygen due to environmental pollution such as ozone depletion weaken the normal function of the skin, accelerate aging, deteriorate blood color, and wrinkle the skin.

To prevent these phenomena, bioactive substances obtained from various known animals, plants, microorganisms, etc. have been added to cosmetics to improve skin by preventing skin aging.

In addition, the cosmetics industry is developing many products using natural ingredients to reduce skin irritation caused by various chemicals. Not only do natural ingredients have fewer side effects on the skin, but their development value as cosmetic ingredients is gradually increasing as consumers are increasingly responding to cosmetics using natural ingredients.

For example, a number of products have been developed that extract active ingredients from germinated sprouts and apply them to cosmetic compositions. These products contain active ingredients such as amino acids and antioxidants, but there was a disadvantage that the active ingredients could not be extracted from the sprout with high efficiency.

In other words, cosmetic products containing extracts of natural origin obtained by conventional methods had the disadvantage of not fully realizing functionalities such as skin improvement effects.

### SUMMARY

Accordingly, the purpose of the present disclosure is to provide a plant-based wrinkle-improving composition and a method for producing the same in which plant seeds are sprouted with a specific wavelength of light in the presence of a photosynthesis promoter to extract only high-quality plant-based amino acids with high efficiency.

Another purpose of the present disclosure is to provide a plant-based wrinkle-improving composition and a method for producing the same in which an amino acid composition with small particle size that easily penetrates the skin is produced, resulting in excellent moisturizing properties and excellent anti-wrinkle effects.

To address the issues, the present disclosure provides a plant-based anti-wrinkle composition comprising plant-based amino acids extracted from sprouted plant sprouts, wherein the sprouted plant sprout is hydroponically sprouted by irradiating red light and blue light in the presence of zinc-sulfur solution manufactured from sphalerite.

The zinc-sulfur solution is prepared from sphalerite.

The red light has a wavelength of 610 to 700 nm and the blue light has a wavelength of 425 to 490 nm,

The sprouted plant sprout is heated and hydrolyzed to remove hard cellulose fibers and extract plant-based amino acids.

The method for preparing a plant-based anti-wrinkle composition comprises step (a) of preparing a plant seed, step (b) of preparing sphalerite (ZnS) powder as a photosynthesis promoter, step (c) of preparing zinc-sulfur solution from the prepared zinc sphalerite powder, step (d) of adding the prepared zinc-sulfur solution to the prepared plant seed to swell the seed, step (e) of first hydroponic cultivating the swelled seed in a dark room, and then second hydroponic cultivating the seed by irradiating the seed with red light and blue light, thereby obtaining a sprouted plant sprout, and step (f) of hydrolyzing an amino acid from the obtained sprouted plant sprout, filtering and separating the amino acid to preparing a plant-based amino acid composition.

In step (f), cellulose is first separated from the obtained sprouted plant sprouts, thereby producing a liquid amino acid-microfiber complex, and the produced amino acid-microfiber complex is heated to hydrolyze amino acids, and then the results are filtered and separated to produce the amino acid composition.

In step (e), the swollen seed is sprayed with the prepared zinc-sulfur solution for 1 to 5 minutes every hour in a dark room, and then the seed is first hydroponically cultivated for 12 to 48 hours, and then second hydroponically cultivated for 12 to 72 hours while irradiating the seed with red light and blue light simultaneously or alternately, thereby obtaining the sprouted plant sprout.

The red light has a wavelength of 610 to 700 nm and the blue light has a wavelength of 425 to 490 nm, the plant seed includes a mung bean or soybean, the particle size of the sphalerite powder is 1 to 100 μm, and the zinc-sulfur solution in step (c) has a zinc standard content of 300 to 9,000 ppm, and the zinc-sulfur solution in steps (d) and (e) is diluted to have a zinc standard content of 30 to 1,500 ppm.

The present disclosure has the advantage of highly efficiently extracting only high-quality plant-based amino acids from sprouted sprouts to have excellent moisturizing and anti-wrinkle effects and to form skin with elasticity, vitality, and volume.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a photograph of sprouted mung bean sprouts according to Examples 1 and 2 of the present disclosure;

FIG. 2 is a photograph of a cross-section of the stem portion of the sprouted mung bean sprouts of FIG. 1;

FIG. 3 is a photograph of sludge after separating amino acid components from amino acid-microfiber complexes of Example 1 of the present disclosure and Comparative Example 1;

FIG. 4 is a photograph of a liquid composition after separating amino acid components from amino acid-microfiber complexes of Example 1 and Comparative Example 1 of the present disclosure;

FIG. 5 is a photograph analyzing the surface moisture state of Example 1 and Comparative Example 1 according to Experimental Example 3 of the present disclosure;

FIG. 6 is a photograph analyzing the wrinkle relief of Example 1 and Comparative Example 1 according to Experimental Example 3 of the present disclosure; and

FIG. 7 is a photograph of sphalerite according to the present disclosure.

### DETAILED DESCRIPTION OF EXEMPLARY EMBODIMENTS

Hereinafter, the present disclosure is described in detail.

There is a problem in that when plant seeds are sprouted by hydroponic cultivation using the conventional method, the cellulose fiber tissue is not strong, so the phase separation rate is low when separating amino acids, making it difficult to obtain high-quality amino acids. This is because the weak cellulose fiber tissue is broken down and mixed together when the amino acids are heat-separated.

Accordingly, the present disclosure has the greatest feature in that when cultivating plant seeds hydroponically, a firm, hard cellulose fiber tissue is generated in a short period of time and soft amino acids are generated and distributed between the fiber tissues, thereby effectively separating only soft amino acids.

To this end, the present disclosure is to provide a plant-based wrinkle-improving composition in which plant seeds are sprouted with a specific wavelength of light in the presence of a photosynthesis promoter to effectively separate and extract only plant-based amino acids, thereby providing a composition with an excellent wrinkle-improving effect. Here, the ‘anti-wrinkle’ means suppressing or reducing the formation of wrinkles on the skin, or alleviating wrinkles that have already been formed.

More specifically, the present disclosure provides a plant-based anti-wrinkle composition comprising plant-based amino acids extracted from sprouted plant sprouts, wherein the sprouted plant sprout is hydroponically sprouted by irradiating red light and blue light in the presence of zinc-sulfur solution manufactured from sphalerite.

That is, when plant seeds containing proteins are hydrolyzed by irradiating them with red and blue light in the presence of zinc-sulfur solution, which is a photosynthetic accelerator, the hard components of the dried seeds absorb water and nutrients in a short period of time, and as they grow through photosynthesis, a solid cellulose fiber tissue is formed. And, as soft amino acids are generated and distributed between these fiber tissues (large molecules of hard proteins change into flexible and fine amino acid units), cellulose is formed as insoluble hard cellulose, and when the amino acids are separated by heating and hydrolysis, the cellulose is not decomposed, and only soft amino acids are separated, thereby obtaining a high-content, small-particle amino acid liquid composition.

The amino acid particles contained in this composition are fine and flexible, and have strong generative performance, so when applied to the skin, they effectively penetrate deep into the skin and supplement fibroblast growth factors to support collagen proliferation and regeneration, thereby alleviating wrinkles and forming elastic, lively, and voluminous skin, as well as assisting the skin's own free amino acids. In addition, anthocyanin and vitamins, which are antioxidant substances produced during photosynthesis, delay skin aging.

In the present disclosure, the red light means light with a wavelength of 610 to 700 nm, and the blue light means light with a wavelength of 425 to 490 nm. By irradiating them simultaneously or alternately, the red light promotes photosynthesis and the growth of the stem length, and the blue light strengthens the stem and makes the leaves lush. That is, by irradiating these specific wavelengths, hard fibrous cellulose is produced through industrially advantageous short-term photosynthesis, and flexible amino acids are finely distributed and produced between the fibrous tissues.

In addition, the present disclosure uses zinc-sulfur solution as a photosynthetic catalyst, and the zinc-sulfur solution is produced from sphalerite.

The present disclosure is to form a strong cellulose fiber tissue in a short period of time to facilitate the separation of amino acids, so zinc and sulfur are important. Since zinc and sulfur are elements that reduce the phenomenon of plant sprouts being crushed during hydroponic cultivation, the present disclosure uses zinc-sulfur solution containing both zinc and sulfur. In addition, zinc and sulfur, which are mixed in small amounts with amino acids during the growth and ingestion of plants, are included in small amounts as cosmetic raw materials, but they extend the storage period of cosmetics and help with antibacterial and UV-blocking effects when applied to the skin. However, this is not the main purpose of the present disclosure. In addition, zinc and sulfur do not cause side effects.

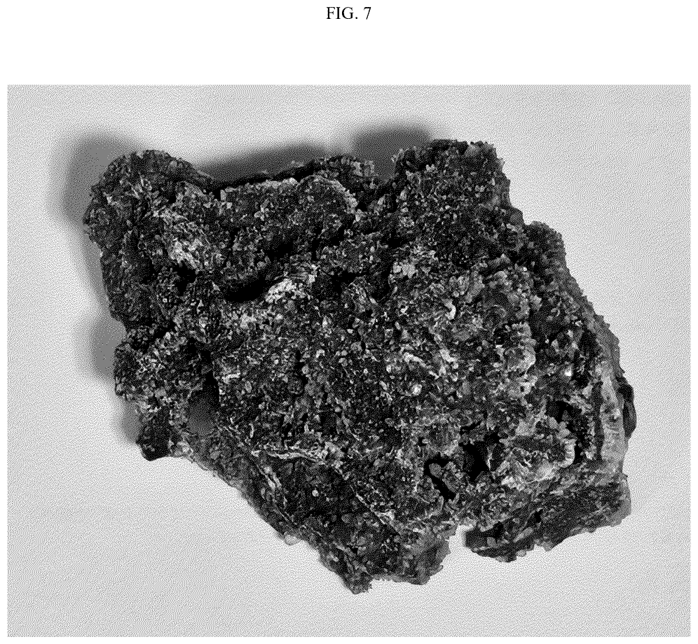

Further, the reason why sphalerite is used in the manufacture of the zinc-sulfur solution is that pure zinc is tough and strong in the process, making it difficult to grind finely, but sphalerite is easily ground by a ball mill to be manufactured into fine particles. In addition, other elements that can be included as natural ores include iron, silica, etc., These are elements that are allowed for human consumption in trace amounts when applied to the human body, so they are harmless to the human body. This is because elements such as As and Pb that may be harmful can be completely removed before use, or purified stone from which harmful substances have been removed or selected stone from which harmful elements do not exist can be used. FIG. 7 is a photograph of sphalerite, and the present disclosure uses purified sphalerite from which harmful substances have been removed.

The amino acid composition obtained from the sprouted plant sprouts hydroponically sprouted by irradiating red light and blue light in the presence of zinc-sulfur solution manufactured from sphalerite is separated by heating and hydrolyzing amino acids from the sprouted plant sprouts in which hard cellulose is formed. Therefore, it has the advantages of excellent separation efficiency, fine particles, flexibility, easy skin penetration, and strong generative performance, imparting excellent moisturizing and anti-wrinkle effects.

Hereinafter, the method for preparing a plant-based anti-wrinkle composition of the present disclosure will be described in detail.

The method for preparing a plant-based anti-wrinkle composition comprises step (a) of preparing a plant seed, step (b) of preparing sphalerite (ZnS) powder as a photosynthesis promoter, step (c) of preparing zinc-sulfur solution from the prepared zinc sphalerite powder, step (d) of adding the prepared zinc-sulfur solution to the prepared plant seed to swell the seed, step (e) of first hydroponic cultivating the swelled seed in a dark room, and then second hydroponic cultivating the seed by irradiating the seed with red light and blue light, thereby obtaining a sprouted plant sprout, and step (f) of hydrolyzing an amino acid from the obtained sprouted plant sprout, filtering and separating the amino acid to preparing a plant-based amino acid composition.

### Step (a) of Preparing a Plant Seed

First, plant seeds are prepared.

In the present disclosure, the plant seeds are not limited, but it is natural to use seeds that contain protein, such as mung beans and soybeans, that is, seeds that are rich in protein and can be grown hydroponically. At this time, the protein content of the seeds is not limited, and for example, 19.0 to 37.7 weight % is sufficient.

### Step (b) of Preparing Sphalerite (ZnS) Powder as a Photosynthesis Promoter

Further, the sphalerite powder is prepared as a photosynthesis promoter. The reason why sphalerite is used as a photosynthesis promoter in the present disclosure is because it is easy to manufacture fine zinc (Zn) and sulfur(S) complex solution by utilizing its brittle nature. The zinc content of the sphalerite is about 40 to 67 weight %.

The particle size of the sphalerite powder is preferably 0.1 to 100 μm, because it is easy to manufacture fine sulfur and zinc complex solution.

In the present disclosure, the fine zinc and sulfur complex solution (hereinafter referred to as ‘zinc-sulfur solution’) acts as an effective fertilizer to increase amino acids and cellulose synthesis through hydroponics within 56 to 110 hours, and is used for the purpose of promoting photosynthesis, amino acid formation, and celluloid formation.

The zinc and sulfur participate in the hydration of carbon dioxide in photosynthesis of plants and also play an important role in the growth of plants. More specifically, zinc is used as an activator of aldolase, which is one of the key enzymes used in the carbon metabolism process of photosynthesis, catalyzing the conversion of carbon metabolism during plant growth and maintaining the structural integrity of dehydro acetone and glyceraldehyde-3-phosphate to fructose 1,6-diphosphate, and can enter the pathway of starch synthesis in chloroplasts. Further, zinc is also necessary for promoting amino acid metabolism during plant growth and maintaining the structural integrity of the ribosomal protein body. When zinc is deficient in plants, the activity of RNA polymerase in plants increases, RNA degradation is accelerated, and amino acid content decreases. In addition, trace amounts of zinc and sulfur can have beneficial effects on human skin, such as antibacterial and UV protection.

Step (c) of Preparing Zinc-Sulfur Solution from the Prepared Zinc Sphalerite Powder

Then, the sphalerite powder is put into a ball mill tank, 1 to 10 times the weight of water and 10 to 50 times the weight of a ceramic ball mill are put in, and then the powder is ground at 40 to 2,000 rpm for 4 to 96 hours to produce zinc-sulfur solution having a zinc standard content of 300 to 9,000 ppm.

As described above, the sphalerite powder is easily broken, so that fine particles of zinc-sulfur solution can be easily produced.

### Step (d) of Adding the Prepared Zinc-Sulfur Solution to the Prepared Plant Seed to Swell the Seed

Next, the prepared zinc-sulfur solution is added to the prepared plant seed to swell the seed for 6 to 48 hours.

At this time, it is preferable to use the zinc-sulfur solution by diluting it so that the zinc standard content is 30 to 1,500 ppm, and the amount used is preferably 1 to 20 times the weight of the prepared plant seeds. This is for sufficient swelling of the plant seeds.

Through this swelling process, sufficient water and nutrients are supplied to the dried plant seeds, enabling rapid cellulose synthesis and amino acid formation.

Step (e) of first hydroponic cultivating the swelled seed in a dark room, and then second hydroponic cultivating the seed by irradiating the seed with red light and blue light, thereby obtaining a sprouted plant sprout

And after placing the swollen seeds in 2-3 layers on the porous tray of the hydroponic cultivation device, hydroponic cultivation is performed in a dark room for 12 to 48 hours. At this time, it is more preferable to spray the diluted zinc-sulfur solution used for the swelling for 1 to 5 minutes every hour.

After hydroponic cultivation in a dark room for 12 to 48 hours, light is irradiated for photosynthesis. At this time, the light with red light of 610 to 700 nm and the light with blue light of 425 to 490 nm are alternately or simultaneously irradiated to obtain sprouted plant sprouts in order to promote plant growth. At this time, the light irradiation time is preferably 12 to 72 hours, because if the time is too short, sufficient photosynthesis is difficult. At this time, the cross-irradiation of the red light and blue light is sufficient if one type of light is applied alternately for 10 minutes each.

The reason why both red light and blue light are irradiated in the present disclosure is that red light is a wavelength that promotes photosynthesis and the growth of the length of the stem, and blue light is a wavelength that strengthens the stem and makes the leaves lush, so that through short-term photosynthesis which is industrially advantageous, hard fibrous cellulose is generated, and flexible amino acids are hydrolyzed and finely distributed between the cellulose fibrous tissues, thereby easily separating only the soft amino acids.

Step (f) of Hydrolyzing an Amino Acid from the Obtained Sprouted Plant Sprout, Filtering and Separating the Amino Acid to Preparing a Plant-Based Amino Acid Composition

And after washing the obtained sprouted plant sprouts with water, they are pressed at a pressure of 1,000 to 10,000 bar to first separate the cellulose, thereby removing the coarse cellulose. At this time, the obtained amino acid-microfiber complex has a solid content of about 5.5 to 41.3%.

Next, the amino acid-microfiber complex is heated at 85 to 125° C. for 40 to 300 minutes. Through this heating process, the amino acid is hydrolyzed and separated from the fiber, and the microfibers are entangled with each other to form a lump. Next, these are filtered and separated to obtain a composition containing 5.8 to 17.3% of transparent reddish brown amino acid (including semi-amino acid components).

The reddish brown composition has excellent generative performance, flexibility, and contains fine-grained plant amino acids, as well as beneficial nutrients such as anthocyanins and vitamins additionally formed through photosynthesis.

Therefore, when this composition is applied to the skin, it reduces wrinkles and delays aging by providing moisturizing properties.

The composition containing the plant-based amino acid of the present disclosure manufactured by the method has an excellent effect on reducing wrinkles, and therefore, it can be used as a plant-based ingredients for cosmetics, health supplements, nutritional supplements, etc. as a composition for anti-wrinkles, and in particular, it can be used as a cosmetic ingredient, applied to the skin surface and penetrated deeply, thereby enhancing its efficacy.

For example, the mixing ratio as a cosmetic composition is as follows: 0.245 to 1.167 weight % of the composition for anti-wrinkles of the present disclosure, 0.020 to 0.060 weight % of adenosine, 0.002 to 0.008 weight % of Lavandula angustifolia (lavender) oil, 0.002 to 0.008 weight % of litsea cubeba fruit oil, 0.001 to 0.005 weight % of Ocimum basilicum (basil) oil, 0.001 to 0.004 weight % of phenoxyethanol, 9.450 to 18.400 weight % of glycereth-26, 6.125 to 12.478 weight % of butylene glycol, 4.416 to 9.334 weight % of glycerin, 3.600 to 6.300 weight % of methylpropanediol, 1.000 to 3.000 weight % of niacinamide, 0.450 to 1.200 weight % of benzyl glycol, 0.250 to 0.900 weight % of glyceryl glucoside, and the remaining purified water. However, this is only an example, and it is obvious that a cosmetic composition can be manufactured by mixing known general cosmetic raw materials in a known mixing ratio.

Hereinafter, the present disclosure will be described in detail through examples.

### Example 1

100 g of mung bean seeds (containing 22.3 weight % of protein) were prepared, and 100 g of purified zinc sphalerite powder (containing 40-67% of Zn, 0% of As, 0% of Pb) as shown in FIG. 7 was prepared as a photosynthesis promoter. The particle size of the sphalerite powder was 1 to 100 μm.

Then, 100 g of the sphalerite powder, 500 g of water, and 3,000 g of a ceramic ball mill were placed in the ball mill tank, and then rotated at 125 rpm for 26 hours to produce zinc-sulfur solution (zinc standard content 730 ppm).

Next, 500 g of the diluted zinc-sulfur solution, which was diluted to 67 ppm of the zinc standard content, was added to 100 g of the prepared mung bean seeds, and the seeds were allowed to swell for 24 hours.

Next, the swollen plant seeds were placed on the porous tray of the hydroponic device in a thickness of 2 to 3 layers, and then the seeds were hydroponically cultivated in a dark room for 28 hours while the diluted zinc-sulfur solution was sprayed for 2 minutes every hour.

After 28 hours, red mung bean sprouts were obtained by irradiating with two wavelengths of red light (610-700 nm) and blue light (425-490 nm) alternately for 10 minutes each for 36 hours.

After washing 100 g of the obtained mung bean sprouts with 10,000 g of water, they were pressurized at a pressure of 3,250 bar to first separate the cellulose fibers, thereby preparing an amino acid-microfiber complex in a water-dispersible liquid phase with a solid content of 21.5%.

Then, the amino acid-microfiber complex was heated at 100° C. for 90 minutes to hydrolyze the amino acid, thereby separating it from the insoluble cellulose fibers. Then, they were filtered and separated again at a pressure of 1,050 bar to prepare a composition containing a transparent reddish brown amino acid component with a solid content of 9.7%.

### Comparative Example 1

100 g of mung bean seeds (containing 22.3 weight % of protein) were prepared, and 500 g of water was added to the 100 g of mung bean seeds and allowed to swell for 24 hours.

Next, the swollen plant seeds were placed in a porous tray of a hydroponic device with a thickness of 2 to 3 layers, then they were hydroponically cultivated in a dark room for 28 hours while the water was sprayed for 2 minutes every hour, thereby obtaining mung bean sprouts.

After washing 100 g of the obtained mung bean sprouts with 10,000 g of water, they were pressurized at a pressure of 3,250 bar to first separate the cellulose fibers, thereby preparing an amino acid-microfiber complex in a water-dispersible liquid phase with a solid content of 21.5%.

Then, the amino acid-microfiber complex was heated at 100° C. for 90 minutes to hydrolyze the amino acid, thereby separating it from the insoluble cellulose fibers. Then, they were filtered and separated again at a pressure of 1,050 bar to prepare a composition containing a transparent yellow-brown amino acid component with a solid content of 1.7%.

### Experimental Example 1

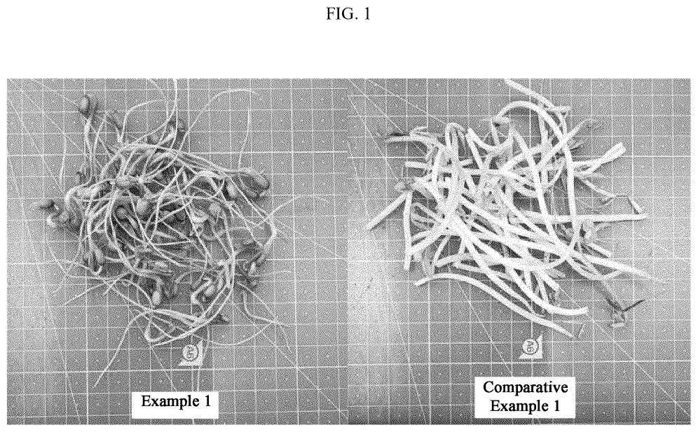

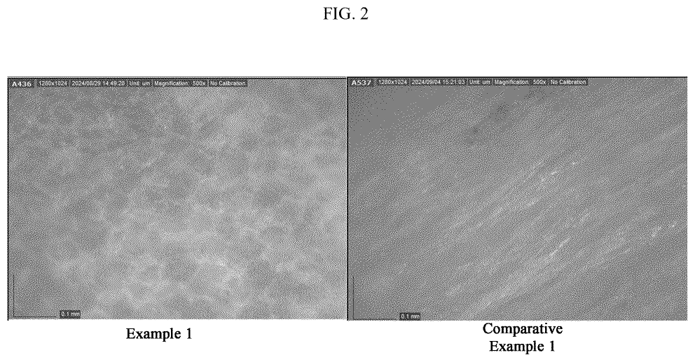

The sprouted shoots of Example 1 and Comparative Example 1 were photographed, and the results are shown in FIG. 1. Further, the stem portion of the sprouted mung beans of Example 1 and Comparative Example 1 was cut with a knife, and the cross-section was photographed using an electron microscope, which is shown in FIG. 2.

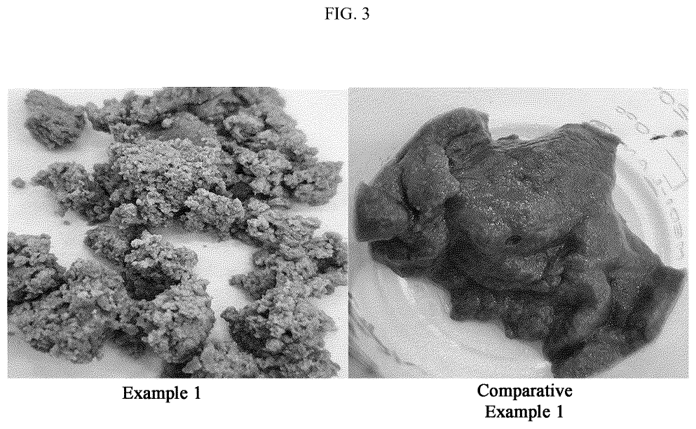

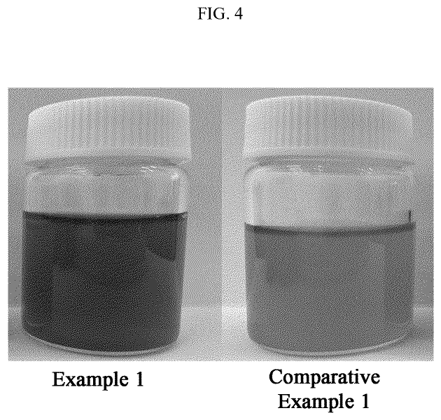

And after separating the amino acid component from the amino acid-microfiber complex of Example 1 and Comparative Example 1 at a pressure of 1,050 bar, the sludge and liquid composition were photographed, and the results are shown in FIG. 3 and FIG. 4.

As shown in FIG. 1, it was confirmed that the sprouted mung bean sprout of Example 1 had a reddish brown color compared to the sprouted mung bean sprout of Comparative Example 1, and as a result of photographing the cross-section, it was confirmed that the sprouted mung bean sprout of Example 1 had a lot of pigment and fiber formed compared to the sprouted mung bean sprout of Comparative Example 1, as shown in FIG. 2.

As shown in FIG. 3, it was confirmed that the sludge of Example 1 had solidly formed fiber, and soft amino acids were separated from hard fiber with a high separation rate. On the other hand, it was confirmed that the sludge of Comparative Example 1 had soft fiber, and soft amino acids were not sufficiently separated, and remained in a mixed state. In addition, as shown in FIG. 4, it was confirmed that Example 1 was able to obtain an amino acid composition with a dark brown solid content of 9.7%, but Comparative Example 1 had a relatively low amino acid separation rate with a solid content of 1.72% and a light color

### Experimental Example 2

5 The liquid compositions obtained from Example 1 and Comparative Example 1 were analyzed for the contents of amino acid components, anthocyanins, vitamins, Zn, Fe, and S using HPLC analysis, and the results are shown in Tables 1 and 2 below.

TABLE 1
Analysis results of component (amino
acid) by Experimental Example 2 (ppm)

| Component (amino acid) | Example 1 | Comparative Example 1 |
|---|---|---|
| Phenylalanine | 1583.0 | 8.5 |
| Valine | 1173.7 | 1347.0 |
| Cysteine | — | 78.0 |
| Aspartic acid | 3783.7 | 72.0 |
| Glycine | 819.8 | 19.0 |
| Serine | 1323.9 | 48.0 |
| Threonine | 878.5 | 7.5 |
| Histidine | 731.4 | 515.0 |
| Tyrosine | 457.9 | 79.0 |
| Alanine | 1075.2 | 14.0 |
| Glutamic acid | 3246.1 | 48.0 |
| Proline | 2002.2 | 81.0 |
| Taurine | — | 106.1 |
| Isoleucine | 963.6 | — |
| Leucine | 1814.8 | — |
| Lysine | 1580.5 | — |
| Methionine | 155.8 | — |
| Arginine | 1547.6 | 61.0 |
| Total | 23237.7 | 2484.2 |

TABLE 2
Analysis results of component (anthocyanins
and the like) by Experimental Example 2 (ppm)

| Component | Example 1 | Comparative Example 1 |
|---|---|---|
| anthocyanins | 5.9 | 0.0 |
| vitamin | 24.7 | 1.3 |
| Zn | 79.8 | 0.2 |
| Fe | 5.2 | 1.7 |
| S | 43 | 2.6 |

As shown in Tables 1 and 2, it was confirmed that Example 1 has a wider variety of amino acids and a richer content than Comparative Example 1, and also has a much higher content of anthocyanins and vitamins. In addition, it was confirmed that Example 1 was rich in zinc and sulfur.

### Formulation Example 1 and Comparative Formulation Example 1

A cosmetic composition was prepared with the mixing amount shown in Table 3 below.

TABLE 3

| | | | Comparative |
|---|---|---|---|
| | | Formulation | Formulation |
| Component | | Example 1 | Example 1 |
| adenosine | Skin-Conditioning | 0.040 | 0.040 |
| | Agent | | |
| lavender oil | Fragrance | 0.004 | 0.004 |
| litsea cubeba fruit oil | Fragrance | 0.004 | 0.004 |
| basil Oil | Fragrance | 0.040 | 0.040 |
| phenoxyethanol | Preservative | 0.002 | 0.002 |
| glycereth | Humectant | 13.500 | 13.500 |
| butylene glycol | Humectant | 8.271 | 8.271 |
| glycerin | Humectant | 6.780 | 6.780 |
| methylpropanediol | Solvent | 4.500 | 4.500 |
| niacinamide | Skin-Conditioning | 2.000 | 2.000 |
| | Agent | | |
| benzyl glycol | Solvent | 0.900 | 0.900 |
| glyceryl glucoside | Humectant | 0.500 | 0.500 |
| Example 1 | — | 0.497 | — |
| Comparative | — | — | 0.497 |
| Example 1 | | | |
| purified water | Solvent | 61.864 | 61.864 |

### Experimental Example 3

A human skin application test was performed using Formulation Example 1 including Example 1 and Comparative Formulation Example 1 including Comparative Example 1 as samples, and then the improvement in surface moisturizing effect and 24-hour persistence effect were analyzed by Epsilon E100, and the results are shown in FIG. 4. Specifically, the moisturizing effect was evaluated by measuring the brightness before, immediately after, and 24 hours after applying the sample to human skin. The brighter the application area appears, the more improved skin hydration can be considered.

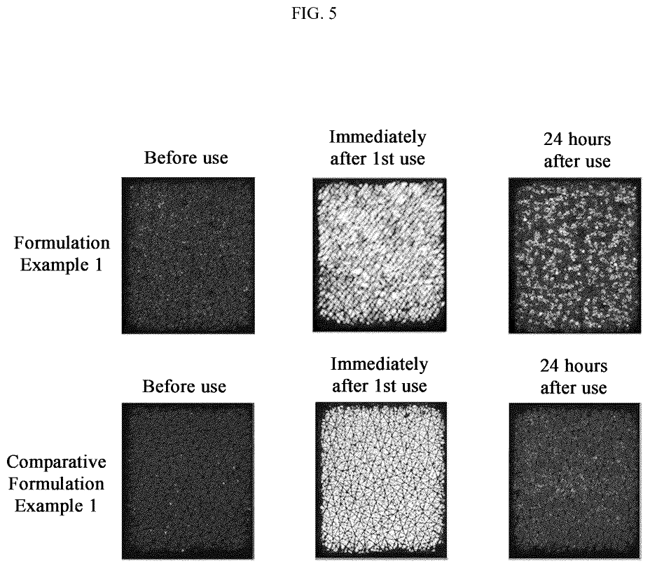

As shown in FIG. 5, it was confirmed that Formulation Example 1 showed an immediate increase in moisturizing rate of about 442% after application, but Comparative Formulation Example 1 showed only a 317% increase, and Formulation Example 1 showed a superior moisturizing effect than Comparative Formulation Example 1 even after 24 hours.

In addition, after washing the face with soap and waiting for 10 minutes in a constant temperature and humidity room (22±2° C., 40˜60% RH), photographs were taken with ANTER 3D, and after using the sample for 2 weeks, photographs were taken again with ANTER 3D in the same manner, and improvement in wrinkles was evaluated based on the photographs taken.

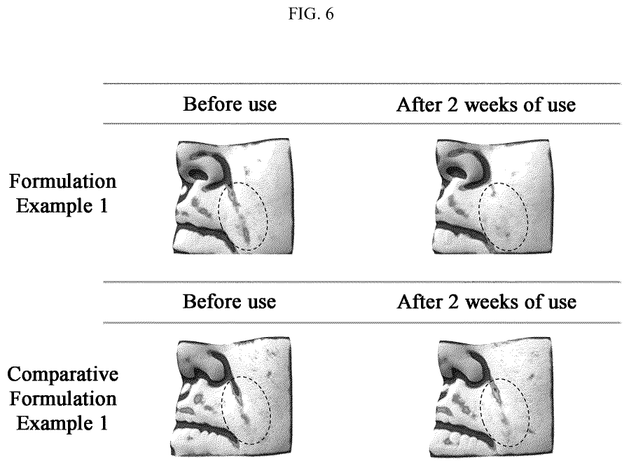

As shown in FIG. 6, Formulation Example 1 including Example 1 alleviated nasolabial folds by about 41.3%, while Comparative Formulation Example 1 including Comparative Example 1 alleviated nasolabial folds by about 22.7%. As a result, the nasolabial fold area became more elastic after 2 weeks of using the sample, and through this, it was confirmed that Formulation Example 1 of the present disclosure had an excellent wrinkle improvement effect.

### Experimental Example 4

A sensory evaluation was conducted on the wrinkle improvement effect and the feeling of use of the cosmetic compositions of Formulation Example 1 and Comparative Formulation Example 1.

Specifically, 10 adults in their 30s to 50s used each cosmetic composition for about 6 weeks, and then evaluated the wrinkle improvement effect, moisturizing effect, and feeling of use on a scale of 1 to 5, and the results are shown in Table 4 below. (1 point: very low, 2 points: low, 3 points: average, 4 points: high, and 5 points: very high)

TABLE 4
Results of Experimental Example 4

| | Wrinkle | | |
|---|---|---|---|
| Classification | Improvement | Moisturizing | Feeling of use |
| Formulation | 4.8 | 4.5 | 3.5 |
| Example 1 | | | |
| Comparative | 3.5 | 3.2 | 3.5 |
| Formulation | | | |
| Example 1 | | | |

As shown in Table 4, it was confirmed that Formulation Example 1 had superior wrinkle improvement and moisturizing effects compared to Comparative Formulation Example 1, and that there was no difference in the feeling of use.

As such, the present disclosure is not limited to the described examples, and it is obvious to those skilled in the art that various modifications and changes can be made without departing from the spirit and scope of the present disclosure. Accordingly, such modifications or variations should be considered to fall within the scope of the claims of the present disclosure.

What is claimed is:

1. A plant-based anti-wrinkle composition comprising plant-based amino acids extracted from sprouted plant sprouts,
wherein the sprouted plant sprout is hydroponically sprouted by irradiating red light and blue light in the presence of zinc-sulfur solution manufactured from sphalerite.

2. The composition of claim 1, wherein the red light has a wavelength of 610 to 700 nm and the blue light has a wavelength of 425 to 490 nm, and
wherein the sprouted plant sprout is heated and hydrolyzed to remove hard cellulose fibers and extract plant-based amino acids.

3. A method for preparing a plant-based anti-wrinkle composition, the method comprising:
step (a) of preparing a plant seed,
step (b) of preparing sphalerite (ZnS) powder as a photosynthesis promoter,
step (c) of preparing zinc-sulfur solution from the prepared zinc sphalerite powder,
step (d) of adding the prepared zinc-sulfur solution to the prepared plant seed to swell the seed,
step (e) of first hydroponic cultivating the swelled seed in a dark room, and then second hydroponic cultivating the seed by irradiating the seed with red light and blue light, thereby obtaining a sprouted plant sprout, and
step (f) of hydrolyzing an amino acid from the obtained sprouted plant sprout, filtering and separating the amino acid to preparing a plant-based amino acid composition.

4. The composition of claim 3, wherein in step (f), the obtained sprouted plant sprout is pressed to first separate cellulose, thereby producing a liquid amino acid-microfiber complex, and the produced amino acid-microfiber complex is heated at 85 to 125° C. for 40 to 300 minutes to hydrolyze amino acids, and then the results are filtered and separated to produce the amino acid composition.

5. The composition of claim 3, wherein in step (e), the swollen seed is sprayed with the prepared zinc-sulfur solution for 1 to 5 minutes every hour in a dark room, and then the seed is first hydroponically cultivated for 12 to 48 hours, and then second hydroponically cultivated for 12 to 72 hours while irradiating the seed with red light and blue light simultaneously or alternately, thereby obtaining the sprouted plant sprout.

6. The method of claim 4, wherein the red light has a wavelength of 610 to 700 nm and the blue light has a wavelength of 425 to 490 nm,
wherein the plant seed includes a mung bean or soybean,
wherein the particle size of the sphalerite powder is 1 to 100 μm, and
wherein the zinc-sulfur solution in step (c) has a zinc standard content of 300 to 9,000 ppm, and the zinc-sulfur solution in steps (d) and (e) is diluted to have a zinc standard content of 30 to 1,500 ppm.
